(19) United States
(12) Patent Application Publication
(10) Pub. No.: US 2012/0135451 A1
(43) Pub. Date: May 31, 2012

(54) Method and Apparatus for Evaluation of Toxicity

(75) Inventor: Rudolf Rigler, St. Sulpice (CH)

(73) Assignee: BIOPHOS SA, Lausanne (CH)

(21) Appl. No.: 13/376,940

(22) PCT Filed: Jun. 8, 2010
(86) PCT No.: PCT/EP10/58001

§ 371 Date: Feb. 15, 2012

Related U.S. Application Data
(60) Provisional application No. 61/184,894, filed on Jun. 8, 2009.

(51) Int. Cl. C12Q 1/02

(52) U.S. Cl. 435/29

(57) ABSTRACT

The present invention pertains to a method for evaluation of the toxicity of compounds comprising the use of spermatozoa. The inventions also relates to a high throughput system for the toxicity evaluation.

The present invention pertains to a method for evaluation of the toxicity of compounds comprising the use of spermatozoa. The inventions also relates to a high throughput system for the toxicity evaluation.

In today's society it is important to assess the toxicity of both known compounds and specifically new compounds not found in nature. Before a compound or a composition is commercialised the toxicity of the compound or compounds contained in a composition must be tested. The exposure a compound will have vis-à-vis a human dictates the amount of toxicology data needed to fulfil various statutory requirements. Toxicity data of compounds is especially important for chemical compounds which are applied on the skin or administered orally or intravenously.

Traditional methods of assessing the toxicity of chemical compounds, such as assessing the mean lethal dose LD50 or mean lethal concentration LC50, use death of animals as an endpoint. Another approach is to observe clear signs of toxicity at one of a series of fixed dose levels often referred to as Fixed Dose Procedures. Toxicity evaluations based on the death of animals as an endpoint are increasingly under public and institutional scrutiny and are being phased out in some jurisdictions in favour of tests such as fixed dose procedures. Yet, the concept and calculation of median lethal dose for comparison purposes is widely applied. In addition to and sometimes in replacement of methods involving use of animals, in vitro testing methods have been developed. In vitro procedures comprise testing the effects of chemical compounds on cultured bacterial or mammalian cells. When using in vitro procedures often a dose response curve is determined and endpoints such as no-observed-adverse-effect-concentration (NOAEC), inhibition concentration 50% (IC50) and total lethal concentration (TLC) are established. Although consuming less funds compared to methods comprising the use of animals, the culture of microbial and cell cultures is both quite time consuming and costly. Although is appears unlikely to wholly replace animal testing when assessing the toxicity of compounds there is a need to develop new toxicity methods which minimise the use of animal testing and in addition requires less labour than in vitro procedures. Thus, the present invention simplifies the generation of toxicology data compared to in vitro testing. In addition, the present invention comprises the use of spermatozoa for assessing the toxicity of compounds. Compared to the use of bacterial and mammalian cells, a spermatozoon is a highly specialised complex multi-organelle cell (eukaryote). Hence, the toxicity data delivered by the present invention may even improve on the in vitro testing.

A further objective with the present invention is to further improve the sensitivity of a toxicity test where the toxicity is correlated to the motility of spermatozoa. According to the method, the motility of the spermatozoa is obtained by providing a digital picture, or a plurality of digital pictures of the spermatozoa timely separated, and applying statistical analysis of the digital pictures. By applying statistical analysis the concentration of the spermatozoa can be kept high while simultaneously measuring the motility accurately. At a concentration of spermatozoa of above about 50 million/ml an increased accuracy/sensitivity of the obtained data is observed. The increased number of spermatozoa improves the statistical analysis and is believed to improve the accuracy of motility and as a consequence improves sensitivity. Preferably, the concentration of spermatozoa in the sample volume/solution is above about 50 million/ml, e.g. above about 55 million/ml, above about 60 million/mi.

A still further objective of the present invention is to reduce the time for assessing the toxicity of the test compounds. By applying statistical analysis the sensitivity is increased over other computerised sperm motility analysers partly due to the fact accurate motility can be measured in solution having a high concentration of spermatozoa. Due to the increased sensitivity the contacting time after incubation of the test compound with the spermatozoa, i.e. the time starting from mixing a solution comprising the test compounds and a solution comprising the spermatozoa, can be kept low, typically below about 20 hours, below about 15 hours, below about 10 hours, such as below about 8 hours, below about 5 hours, below about 3 hours, below about 1 hour, and even as short as below about 30 minutes.

The present invention relates to a method for evaluation of the toxicity of compounds. The present invention also embraces an apparatus. For added clarity, the compound of which the toxicity is assessed is hereinafter referred to as test compound.

More specifically, the invention relates to a method for evaluating the toxicity of test compounds comprising providing a test compound and spermatozoa, forming solutions (A) comprising the test compound in varying concentrations, forming a solution (B) comprising spermatozoa, forming solutions (C) comprising the solution (A) and (B) and incubating solution (C), whereby the toxicity of the test compound is correlated to the motility of the spermatozoa, wherein the solutions (A) are formed by serial dilution. The motility is preferably obtained by digital image recognition. According to an embodiment the motility is obtained by providing a digital picture of the solution and applying statistical analysis of the digital picture. The digital picture which may be captioned by any type of solid state image sensor, such as charged coupled devices (CCD) or complementary metal oxide semiconductor detectors (CMOS), is reproducing a moving digital picture of the solution comprising test compound and the spermatozoa. The moving digital picture may also be referred to as a video picture. Suitably, the motility is obtained by applying statistical analysis on the digital picture, i.e. on the data (events) of the digital picture. Fluctuation analysis applied on the digital picture is a specifically preferred statistical analysis method. More specifically, correlation analysis of temporal fluctuations of the spermatozoa of the digital picture is applied, and suitably a time correlation function or functions is/are calculated on the temporal fluctuations of the spermatozoa. According to a preferred embodiment the motility is obtained by providing a digital picture of the solution, whereby correlation analysis on temporal fluctuations of the spermatozoa of an area of the digital is applied and a time correlation function(s) is/are calculated based on temporal fluctuations of the spermatozoa. Further details regarding obtaining motility data from a digital picture is evident from WO 2006/074965 which content is herein incorporated by reference. The apparatus for evaluating the toxicity of compounds comprising a sample compartment comprising a solution comprising spermatozoa and at least a test compound, means for generating a digital picture of the solution, computational means for generating motility data of the spermatozoa from the digital picture, wherein the toxicity is based on the motility data of the spermatozoa. The motility is preferably obtained by applying statistical analysis of the digital picture such as fluctuation analysis. Suitably, the motility is obtained by any of the methods as disclosed above. Additional embodiments of the present invention are defined by the claims.

By toxicity is herein meant any relationship between the dose of a compound and its effect on an exposed organism. The organism employed in the present invention is a spermatozoon. Usually, a multitude of spermatozoon is referred to as spermatozoa. Spermatozoa are often one constituent of semen, the latter an organic fluid also known as seminal fluid secreted by the sexual glands and other sexual organs of males. For the purpose of the present invention any solution comprising spermatozoa can be used. Typically, the solution comprising spermatozoa is aqueous. The aqueous solution comprising spermatozoa can be undiluted semen, provided spermatozoa are present in the semen. However, often the semen is diluted prior to use. According to the present invention the solutions (A) comprising the test compound are formed by serial dilution. Serial dilution is a stepwise dilution of a substance in solution. Typically, the dilution factor at each step is constant resulting in a geometric progression of the concentration in a logarithmic fashion. Any dilution factor can be chosen such that a dose response curve is obtained and providing relevant Additionally, the sample solution comprising spermatozoa, e.g. diluted semen, and the test compound may contain further additives which are advantageous for the method. By sample solution is meant the solution comprising spermatozoa and the compound to be evaluated. Such additional compounds include buffer solutions optionally containing polar aprotic solvents, polar aprotic solvents such as dimethyl sulfoxide (DMSO), dimethylformamide, dimethylacetamide, N-methyl-2-pyrrolidone, hexamethylphosphoramide (HMPA), and the like. DMSO is a preferred polar aprotic solvent. Furthermore, other compounds important for the maintenance of the biological and metabolic activity of the spermatozoa are pH buffering agents such as phosphates or 4-(2-hydroxyethyl)-1-piperazineethanesulfonic acid (HEPES) to mention a few, salts such as sodium chloride (NaCl) or potassium chloride (KCl) to maintain the ionic strength of the solution, divalent cations such as Ca²⁺ and Mg²⁺ which is important for maintaining the activity of important proteins and enzymes, and antibiotic agents such as penicillin, streptomycin, lincomycin, and spectinoycin to mention a few. Additional adjuvants which may be present in the sample solution are compounds permeabilising cell membranes, e.g. compounds comprising sugar moieties such as glucose, fructose, glucuronic acid, suitably glycosides exemplified by digitonine. The concentration of spermatozoa in the sample solution may vary within wide limits and may depend on the type of spermatozoa. Typically, the concentration of spermatozoa is above 20 million spermatozoa (cells)/ml based on total sample solution. The upper limit may depend on several factors such as the presence of non-sperm debris. Typically, the concentration of spermatozoa is from about 20 million/ml up to about 150 million/ml, such as from about 20 million/ml up to 120 million/ml. Suitably, the concentration of spermatozoa is above about 50 million/ml, more preferably above about 55 million/ml, above about 60 million/ml. Polar aprotic solvents are added to the sample solution at an amount of from about 0.5% w/w up to about 5% w/w.

The spermatozoa used in the present invention may origin from animals and humans. If diluted semen is used which has not been subjected to removal of debris, e.g. non-spermatozoa aggregates such as proteins, cells and the like. Preferably semen is used having an overall low natural content of debris. Furthermore, the spermatozoa are preferably genetically stable. According to an embodiment of the present invention animal spermatozoa is made use of. Suitably, the spermatozoa may originate from domesticated animal including but not limited to rodents, bovinae, equine, boar, canidae, lepordiae, fish. Spermatozoa from boar species are specifically suited.

It has been found that spermatozoon is a biological entity which is well suited for assessing the toxicity of compounds. Preferably, the compound should be dispersible in an aqueous solution or water-soluble, or rendered dispersible or water-soluble. The toxicity of any chemical compound may be evaluated by the method or apparatus of the present invention. Examples of compounds are naturally occurring compounds which have not been widely used commercially or not used commercially and new compounds such as non-naturally-occurring new chemical entities, the latter synthesised by inter alia the chemical industry including pharmaceutical and agrochemical companies.

The toxicity is evaluated by measuring the motility of the spermatozoa as a function of the concentration of the compound to be evaluated. Based on the dose response of the motility of the spermatozoa data correlating to the toxicity of the compound is obtained. By motility is understood any data associated with the movement of spermatozoa. The motility can be expressed as the share of total motile spermatozoa (i.e. all moving spermatozoa) compared to total amount of spermatozoa. Also the share of a subgroup of the motile spermatozoa to total spermatozoa may be used. Suitable subgroups are groups including spermatozoa having the highest motility, e.g. the share of spermatozoa having a motility above a pre-defined threshold. According to an embodiment, the motility is based on the group of spermatozoa having a motility above about 25 μm/seconds, more preferably above about 30 μm/sec, such as above about 35 μm/seconds. Alternatively, the motility can be expressed as the mean speed of the motile spermatozoa. Regardless of motility parameter chosen a dose response curve is obtained and a suitable endpoint is calculated. Exemplified endpoints include no-observed-adverse-effect-concentration (NOAEC), inhibition concentration 50% (IC50), total lethal concentration (TLC), just to mention a few. Based on the response curve a representative endpoint is easily calculated using standard mathematical procedures.

According to the present invention the motility may be obtained by providing a digital picture of the solution comprising spermatozoa and a test compound and analysing the digital picture by computational means. Any method of digital imaging analysis may be applied on the digital picture provided reliable and reproducible motility data is generated. Examples of digital imaging analysis include trajectory analysis related to mean square particle displacement. The motility of the spermatozoa is preferably generated by a method and apparatus which is able to deliver motility data based on simultaneous measuring a significant number of individual spermatozoa. Motility data which is based on a significant number of spermatozoa is simply more reliable from a statistical point of view, compared to motility data based on just a few spermatozoa. In order to generate reliable data the concentration of spermatozoa in the sample solution should preferably be above about 20 million spermatozoa/ml based on total sample solution, preferably above about 30 million/ml, even more preferably above above about 50 million/ml, above about 55 million/ml and above about 60 million/ml. The upper limit of the concentration of spermatozoa in the sample solution is not critical but is usually below about 120 million/ml, suitably below about 80 million/ml. According to a preferred embodiment the motility is generated by a method comprising statistical analysis such as fluctuation analysis. By applying fluctuation analysis the motility data is based on a high number of spermatozoa. Preferably, the fluctuation analysis generating the motility comprises applying correlation analysis on temporal fluctuations of the spermatozoa with respect to a detection area of a digital picture. More specifically, the motility data is generated by applying correlation analysis on temporal fluctuations of the number of spermatozoon with respect to a detection area of a digital picture, wherein a time correlation function is calculated based on the temporal fluctuations of the number of spermatozoa. Thus, temporal fluctuations of spermatozoa in a digital moving picture are analysed by using correlation analysis which generates a correlation function or correlation functions, said correlation function(s) giving information about dynamic parameters of the particles such as the motility, diffusion times, translation speed, rotational frequency, etc. The digital moving picture of the sample solution comprising the spermatozoa and the compound of interest is generated by inter alia using a suitable detector capable of generating a digital picture such as an image sensor connected to computational means. The digital picture may suitably by computational means be divided into several measurement/detection areas capable of generating adequate time dependent (temporal) fluctuation of the number of spermatozoa or concentration of the spermatozoa over the detection area boundary. Thus, the number of spermatozoa in the detection area is randomly changing around the average number. The fluctuation of the number of spermatoza with respect to a detection area of the digital picture is analysed by the correlation function of the fluctuation signal, the signal representing the spermatozoa. Hence, the fluctuations are the basis for calculating a (time) correlation function or (time) correlation functions for generating the dynamic parameters such as the motility. Over time the spermatozoa will move in the digital picture, that is in-out motion across the detection area boundary. An image sensor of the present invention is a sensor capable of generating digital images of moving particles having a spatial resolution in the image plane, i.e. in x- and y-axis. As the image sensor generates a series of digital images (frames) having a resolution in time, a moving picture is obtained which is further analysed by applying correlation analysis.

According to an embodiment of the present invention the method does not only comprise applying statistical analysis of the digital picture for generating the motility data but also other digital imaging recognition methods which are capable of providing motility data such as trajectory analysis and mean square particle displacement analysis.

The apparatus for assessing the toxicity of chemical compounds comprises a sample compartment comprising spermatozoa and at least a compound, a light source, an image sensor, computational means for processing signals from the image sensor, detecting the spermatozoa on a digital picture generated by the image sensor and calculating at least the motility of the spermatozoa by statistical analysis. According to a preferred embodiment of the present invention the evaluation of the toxicity is performed by an apparatus enabling a high-throughput analysis. Such a high-throughput system comprises modules allowing a high degree of automation capable of assessing the toxicity of a high number of compounds in any given time. Thus, the present invention provides for a high-throughput system for generation of toxicity data of chemical compounds, comprising providing at least a means for storing a sample solution comprising spermatozoa and a chemical compound of which the toxicity is to be assessed, the system comprising means enabling a high rate per time of evaluated samples, wherein the toxicity of the compound is determined by the motility of the spermatozoa. Suitably, the high-throughput system is designed around a chamber comprising a multitude of wells such as microwell chamber containing 96 well or a multiple of 96 well, e.g. 192 well chamber, 288 well chamber, 384 well chamber, etc. Each well contains a sample solution comprising spermatozoa and a chemical compound of which the toxicity is evaluated.

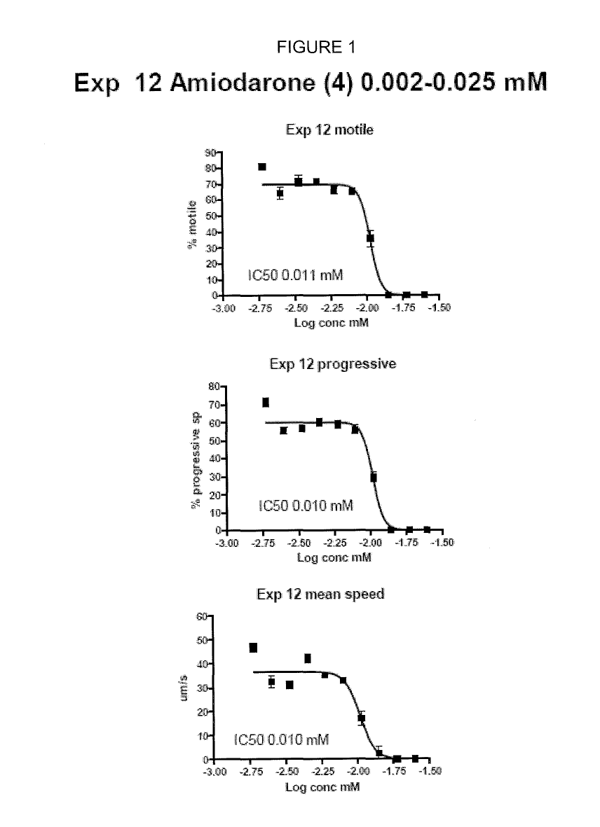

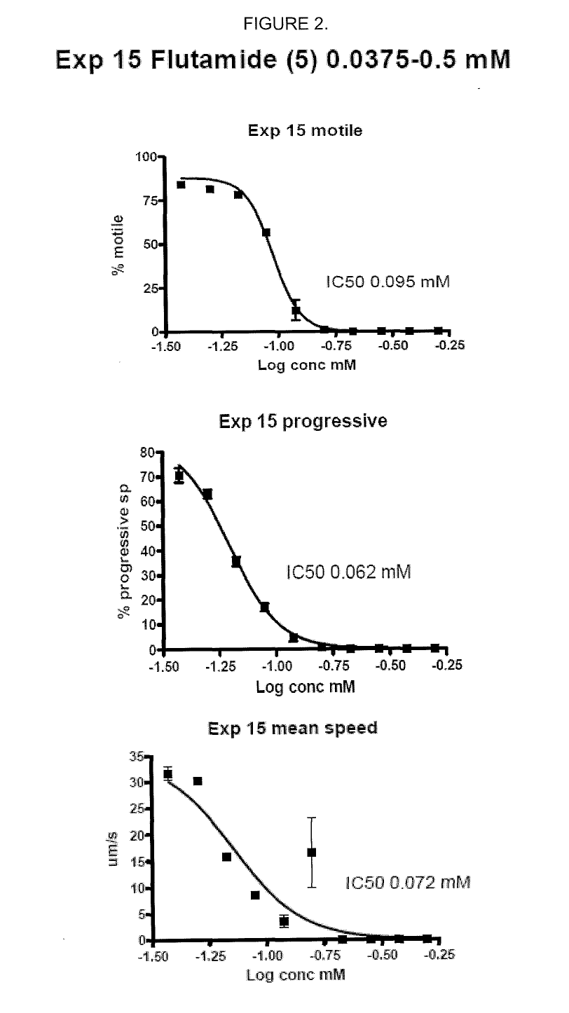

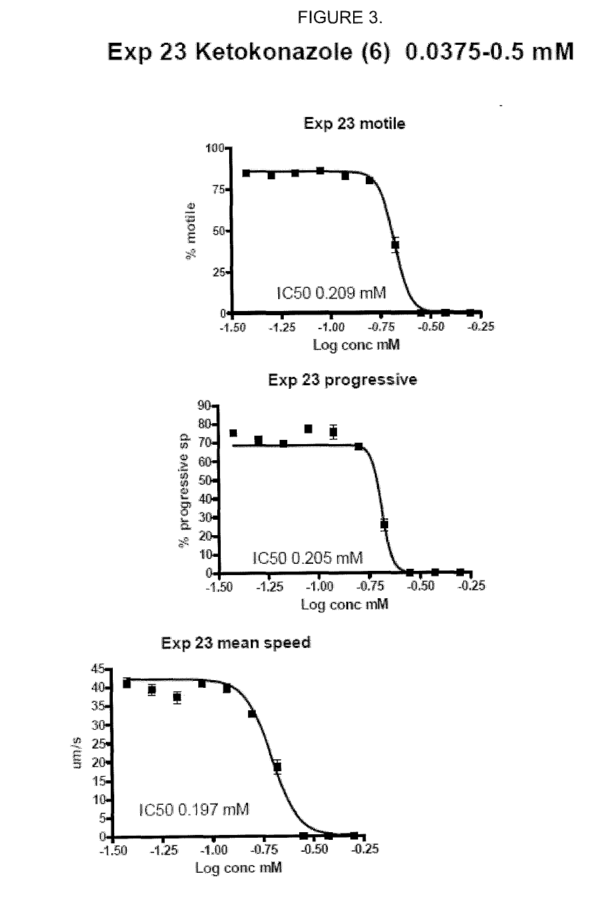

FIGS. 1 to 3 show the dose response curves obtained by using Qualisperm®.

The invention is further exemplified, yet, without the intention to be limiting.

The toxicicity of known toxic compounds was evaluated by measuring the motility of spermatozoa. The following toxic compounds were tested: Amiodarone, Flutamide and Ketoconazole. All of them belong to the group of mitochondrial toxins and hepatotoxins. Non toxic compounds did not show changes in motility of spermatozoa.

Quadruple samples of each test compound were prepared in varying concentrations by serial dilution in a 96 microwell-plate by serial dilution into XCell with DMSO. Equal volumes (50 μl) of boar spermatozoa in a concentration of around 60 million/ml were added to all sample solutions. Subsequently, the microwell plate was shaken and incubated for 30 minutes. The dosing was performed by using an automated TECAN® robot. After the incubation the sample solutions were pipetted on a Leja® slide and the motility of the spermatozoa was analysed using a Qualisperm® apparatus from Biophos AG. A dose response curve was obtained where % motile spermatozoa (all the moving spermatozoa), % progressive spermatozoa (share of spermatozoa above a pre-determined threshold, here: above 25 μm/sec), or mean speed of total motile spermatozoa was plotted as a function of the logarithmic concentration of the test compound expressed in mM. From the dose curve IC50 values were calculated.

Table 1 below shows the IC50 values calculated from the dose response curves.

| | Qualispemr ® | | IC50 % | IC50 % | IC50 mean |
|---|---|---|---|---|---|
| Name | Max Conc | | motille | progressiv | speed |
| Amiodarone | 0.025 | mM | 0.011 | 0.010 | 0.010 |
| Flutamide | 0.5 | mM | 0.095 | 0.062 | 0.072 |
| Ketokonazole | 0.5 | mM | 0.209 | 0.205 | 0.197 |

1. Method for evaluating the toxicity of test compounds comprising providing a test compound and spermatozoa, forming solutions (A) comprising the test compound in varying concentrations, forming a solution (B) comprising spermatozoa, forming solutions (C) comprising the solution (A) and (B) and incubating solution (C), whereby the toxicity of the test compound is correlated to the motility of the spermatozoa, wherein the solutions (A) are formed by serial dilution.

2. The method according to claim 1, wherein the motility is obtained by digital image recognition.

3. The method according to claim 1, wherein the toxicity is correlated to the spermatozoa having a motility of above about 25 μm/seconds.

4. The method according to any one of claims 1 to 3, wherein the motility is obtained by providing a digital picture of the solution and applying statistical analysis of the digital picture.

5. The method according to any one of claims 1 to 4, wherein the duration of the incubation is less than about 15 hours, preferably less than about 10 hours, more preferably less than about 8 hours.

6. The method according to claim 5, wherein the duration of the incubation is less than 1 hour.

7. The method according to any one of claims 4 to 6, wherein the statistical analysis is fluctuation analysis.

8. The method according to any one of claims 4 to 7, wherein correlation analysis of temporal fluctuations of the spermatozoa of the digital picture is applied.

9. The method according to any one of claims 4 to 8, wherein correlation analysis on temporal fluctuations of spermatozoa of the digital picture is applied, and a time correlation function is calculated based on the temporal fluctuations of the spermatozoa.

10. The method according to any one of claims 4 to 9, wherein correlation analysis on temporal fluctuations of spermatozoa of an area of the digital picture is applied, and a time correlation function is calculated based on the temporal fluctuations of the spermatozoa.

11. The method according to any one of the preceding claims, wherein the spermatozoa is animal spermatozoa.

12. The method according to claim 11, wherein the spermatozoa is boar spermatozoa.

13. The method according to any one of the preceding claims, wherein the concentration of spermatozoa in the solution (C) is above about 50 million spermatozoa/ml.

14. The method according to claim 13, wherein the concentration of spermatozoa in the solution (C) is above about 55 million/ml.

15. A high-throughput system for evaluating the toxicity of test compounds comprising a sample compartment comprising a solution comprising spermatozoa and at least a test compound, means for generating a digital picture of the solution, computational means for generating motility data of the spermatozoa from the digital picture, means for enabling a high rate of evaluated compounds, the toxicity being based on the motility data of the spermatozoa, wherein the motility data is obtained by applying statistical analysis of the digital picture

16. The apparatus according to claim 15, wherein the motility data is obtained by fluctuation analysis of the digital picture.

17. The apparatus according to any one of claims 15 to 16, wherein the motility data is obtained by correlation analysis of temporal fluctuations of the spermatozoa of the digital picture.

18. The apparatus according to any one of claims 15 to 17, wherein the motility data is obtained by correlation analysis on temporal fluctuations of spermatozoa of an area of the digital picture, and a time correlation function is calculated based on the temporal fluctuations of the spermatozoa.
